(19) United States
(12) Patent Application Publication
(10) Pub. No.: US 2021/0378950 A1
(43) Pub. Date: Dec. 9, 2021

(54) COMBINATION OF CANNABIS, DERIVATIVES THEREOF AND ADDITIVES IN TOPICAL COMPOSITIONS

(71) Applicant: THERESE MAINELLA, NORTHPORT, NY (US)

(72) Inventor: THERESE MAINELLA, NORTHPORT, NY (US)

(21) Appl. No.: 16/892,702

(22) Filed: Jun. 4, 2020

(51) Int. Cl. A61K 9/00; A61K 9/06; A61K 31/167; A61K 31/573; A61K 47/10; A61K 47/46; A61K 47/44

(52) U.S. Cl. CPC A61K 9/0014; A61K 9/06; A61K 31/167; A61K 47/44; A61K 47/10; A61K 47/46; A61K 31/573

(57) ABSTRACT

A topical composition comprising a therapeutically-effective amount of at least one cannabinoid and at least one pharmaceutical active component in which the at least one pharmaceutical active component reduces pain and inflammation beyond the benefits provided by the at least one cannabinoid alone. The pharmaceutical active components can be lidocaine, Manuka honey or Stachys byzantina. The topical composition can be in the form of a lotion, cream, stick or rub.

### BACKGROUND OF THE INVENTION

### Field of the Invention

The present invention relates to topical compositions including materials derived from the cannabis plant and additives to reduce inflammation, antibacterial, antiseptic properties and provide pain relief. The topical compositions can be of a form, for example, of a lotion, gel, and/or rub to apply the topical composition directly on skin surfaces.

### Description of Related Art

Cannabinoid topical compositions have been described. U.S. Pat. No. 9,095,563 describes a topical treatment incorporating an extract of Cannabis sativa or Cannabis indicia. The concentration of cannabidiol in the topical formulation is greater than 2 milligrams per kilogram. The topical preparations can be used to treat dermatological diseases.

U.S. Pat. No. 10,588,979 describes a topical composition including a terpene blend and cannabidiol (CBD) or a derivative thereof, useful for managing pain, the treatment or prevention of inflammatory skin disorders. The composition includes 2-pyrrolidone-5-carboxylic acid and a blend of antioxidants to optimize tissue health.

It is desirable to provide an improved topical composition including a combination of an active component of a cannabis material with a specified combination of active ingredients to provide additional benefits in reducing pain and inflammation beyond the benefits provided by the cannabis related material alone.

### SUMMARY OF THE INVENTION

According to an aspect of the present invention there is provided a topical composition comprising a therapeutically-effective amount of at least one cannabinoid and at least one pharmaceutical active component in which the at least one pharmaceutical active component reduces pain and inflammation beyond the benefits provided by the at least one cannabinoid alone.

Optionally, the cannabinoid is selected from cannabidiol (CBD), cannabigerol (CBG) and cannabichromene (CBC), and Cannabidiphorol (CBDP). In one embodiment the cannabinoid comprises CBG Oil and CBD Oil.

Optionally, the at least one pharmaceutical active component can include one or more pain relief ingredients. The pain relief ingredients can be a local anesthetic numbing medication which work by blocking nerve signals in the body to reduce pain or discomfort. In one embodiment the pharmaceutical active component is Lidocaine.

Optionally, the at least one pharmaceutical active component is Manuka honey. Manuka honey provides anti-bacterial and anti-inflammatory properties.

Optionally, the at least one pharmaceutical active component is Stachys byzantina. Stachys byzantina provides antiseptic, anti-inflammatory and anti-bacterial properties.

Optionally, the at least one pharmaceutical active component is kava. Kava can provide anesthetic and anti-inflammatory and anti-bacterial properties.

Optionally, the topical composition can comprise a butter or wax material. Optionally, the topical composition can comprise one or more oils, water or alcohols.

Optionally, the pharmaceutical active component can include one or more vitamins, minerals, extracts, tocopherols, and herbs.

Optionally the topical composition can comprise one or more additives selected from diluents, preservatives, humectants, thickeners, emulsifiers, emollients, surfactants, and mixtures thereof.

Optionally, the topical compositions can be of a form, for example, of a lotion, gel, stick and/or rub to apply the topical composition directly on a skin or hair surface.

According to an aspect of the present invention there is provided a topical composition comprising a therapeutically-effective amount of at least one in which the at least one pharmaceutical active component reduces pain and inflammation, and optionally is an antiseptic or anti-bacterial, in which the pharmaceutical active component is a local anesthetic and a plant or extract of Stachys byzantina.

The present invention is also directed to methods to prepare a topical product and methods to administer the topical product to a human subject.

### DETAILED DESCRIPTION

It will be readily understood that the components of the present invention, as generally described herein, could be arranged and designed in a wide variety of different configurations or formulations. Thus, the following more detailed description of the embodiments of the system, products and methods of use of the present invention, are not intended to limit the scope of the invention, as claimed, but is merely representative of various embodiments of the invention.

In one embodiment, a topical composition can include a therapeutically-effective amount of at least one cannabinoid and at least one pharmaceutical active component. The pharmaceutical active component can be comprised of any suitable materials. Generally, a pharmaceutical active component provides additional benefits to help improve and/or maintain pain relief and anti-inflammation benefits beyond the basic benefits provided by a cannabinoid. Any number of compounds or materials can be utilized to comprise a pharmaceutical active component of a topical product. In one embodiment, the pharmaceutical active component comprises at least one pharmaceutical pain reducing component and at least one anti-inflammatory component as active components.

Cannabinoids used in the topical care composition can be in liquid form, as a natural constituent of hemp oil or cannabis oil. Hemp oil or cannabis oil is harvested by cold pressing the seeds and the plants of the Cannabis sativa species, as described in US Patent Application Publication No. 2019/0076349, hereby incorporated by reference into this application. The resulting oil is extracted using CO₂ extraction or solvent extraction process and can be further concentrated by distillation. Targeted cannabinoids include cannabidiol (CBD), cannabigerol (CBG) and cannabichromene (CBC) and Cannabidiphorol (CBDP) and cannabinol (CBN). In some embodiments, the cannabinoids can include tetrahydrocannabinol (THC) and tetrahydrocannabiphorol (THCP). The topical composition can also include dried cannabis.

In one embodiment, the at least one pharmaceutical pain reducing component is a local anesthetic which reduces sensation or pain by blocking nerve impulses that send pain sensations to the brain. Suitable pharmaceutical active components can include, without limitation, lidocaine, benzocaine, bupivacaine, mepivacaine and articaine.

In one embodiment, the pharmaceutical active component can include one or more anti-inflammatory ingredients. Suitable compounds or materials in a pharmaceutical active component can include an anti-inflammatory, anti-bacterial or anti-microbial component. In one embodiment, the pharmaceutical active component is an anti-microbial honey. The anti-microbial honey can be Manuka honey which is a type of honey native to New Zealand. Manuka honey is produced by bees who pollinate the flower Leptospermum scoparium, commonly known as the Manuka bush. Manuka honey features antibacterial properties. Methylglyoxal is an active ingredient in Manuka honey and is likely responsible for the antibacterial effects. Manuka honey, without limitation, has antiviral, anti-inflammatory, antioxidant benefits, wound healing and preventative therapy properties.

In one embodiment, the pharmaceutical active component is a plant or extract from a plant of the genus Stachys or of the lamiaceae family. In one embodiment the plant or extract is Stachys byzantina, also referred to as lamb's ear plant. Stachys byzantina plant and extract, without limitation, has antiseptic, anti-inflammatory and anti-bacterial properties.

In one embodiment, the at least one pharmaceutical active component is kava. Kava can be made from the root or stump of the kava the Piper methysticu shrub. Kava is a member of the pepper family of Piperaceae, Kava can be used as a plant, root or extract from the plant or root. Kava can be in powdered form. Kava can be in the form of kava lactones found in the roots and rhizomes. Kava plant and extract, without limitation, has anesthetic antiseptic, anti-inflammatory and anti-bacterial properties. An effective daily dose of kava can range from 70 mg to 200 mg of kavalactones,

In one embodiment, the topical composition can be formulated into a lotion, cream, rub or ointment. Suitable ingredients for the lotion, cream, rub or ointment include water, thickeners and oils. In some embodiments, the thickeners include, without limitation, natural cocoa butter, bees wax, shea butter, candelilla vegan wax, and wax. In some embodiments, the oils include, without limitation one or more of coconut oil, olive oil, avocado oil, mineral oil, and sunflower seed oil. In one embodiment the topical composition includes aloe vera gel. In some embodiments, the topical composition includes petroleum jelly. In some embodiments, the topical composition includes one or more of cetyl alcohol, stearyl alcohol and methylcellulose. In some embodiments, the topical formulation includes an alcohol, including, without limitation, benzyl alcohol or isopropyl alcohol.

In some embodiments of the present invention, the topical composition comprises a pharmaceutical active component of a steroid to treat a skin condition. Example skin conditions can include redness, swelling, itching, and discomfort. The topical composition can include corticosteroids including hydrocortisone. Suitable steroids which can be used in the topical composition include clobetasol and halobetasol.

In some embodiments, the pharmaceutical active component can include one or more vitamins or minerals. Vitamins can include, for example, vitamin E, D, B12. product. In one embodiment, the additives can include herbal supplements including without limitation, cayenne, capsicum, capsaicin, and capsaicinoid compounds. In some embodiments, the pharmaceutical active component can include one or more extracts, herbs and spices to supplement or improve health properties of an the topical product. For example, the pharmaceutical active component extracts can include vegetable and fruit extracts, as well as stem cells of plants, including, without limitation, peppermint oil, tea tree oil, eucalyptus oil, chamomile oil, neem oil, lavender oil, garlic extract, neem extract, marigold extract and oregano extract. The adaptogenic herbs can include, without limitation, menthol, lavender, rose hip, rose petals, fennel, and tea leaves.

In some embodiments of the present invention, the topical formulation further comprises an antifungal agent. The antifungal agent is not particularly limited, and is preferably at least one of ketoconazole, bifonazole, butoconazole, clotrimazole, econazole, fenticonazole, isoconazole, luliconazole, miconazole, omoconazole, oxiconazole, sertaconazole, sulconazole, tioconazole, albaconazole, fluconazole, isavuconazole, itraconazole, posaconazole, ravuconazole, terconazole, and voriconazole,

In some embodiments, the topical composition can comprise one or more additives to provide desired properties of a topical product. In some embodiments, the topical composition comprises one or more of aluminum sulfate, lactic acid neutralized with ammonium hydroxide, magnesium aluminum silicate, and laureth-4, polyoxyl 40 stearate.

In a still further embodiment, the topical composition can comprise at least one emollient, surfactant and/or emulsifier as an additive. Any acceptable emollient, surfactant or emulsifier can be used. Suitable emollients, include polyethylene glycol ester (PEG) of stearic acid, PEG-100 Stearate, and glyceryl stearate and the like.

In some embodiments, the additives can include humectants including, without limitation, propylene glycol, vegetable glycerin and glycerin methyl paraben. In some embodiments, sodium parabens are used as preservatives. Suitable parabens include parahydroxybenzoates or esters of parahydroxybenzoic acid, methyl ethylparaben, propylparaben, and the like.

In some embodiments, the additives can include sunscreen agents. The sunscreen agent is not particularly limited, and is preferably at least one of aminobenzoic acid (PABA), avobenzone, cinoxate, dioxybenzone, ensulizole, homosalate, meradimate, octinoxate, octisalate, octocrylene, padimate O, sulisobenzone, oxybenzone, trolamine salicylate, ethylhexyl methoxycinnamate, octyl methoxycimnamate, octyl salicylate, phenylbenzimidazole sulfonic acid, titanium dioxide, trolamine dioxide and zinc oxide.

In a still further embodiment, the topical composition of the invention can comprise at least one colorant as an additive. Colorants herein include pigments, dyes and agents.

The composition can comprise from about 0.1% to about 60% by weight or by volume of the cannabinoid, for example, 1.0-50%, 5-45%, 10-40%, 20-40%, 25-40%, or 30-40% by weight or by volume.

The composition can comprise from about 0.1% to about 45% by weight or by volume of the pharmaceutical or otherwise active agent, for example, 0.1-1.0%, 0.1-0.9%, 0.1-0.8%, 0.1-0.7%, 0.1-0.6%, 0.1-0.5%, 0.1-0.3%, 0.1-0.2%, 1%-45%, 5%-40%, 10-35%, 10-30%, 15-30.%, 20-30% or 25-30% by weight or by volume. In one embodiment the pharmaceutical or otherwise active agent is a pain reduction or relief agent which is present in an amount of 1 to 40%, 1 to 30%, 5-40%, 10-35%, 10-30%, 15-30.%, 20-30% or 25-30% by weight or by volume. In one embodiment the pharmaceutical or otherwise active agent is Manuka honey which is present in an amount of 0.5 to 40%, 1 to 30%, 5-40%, 10-35%, 10-30%, 15-30.%, 20-30% or 25-30% by weight or by volume. In one embodiment the pharmaceutical or otherwise active agent is Stachys byzantina which is present in an amount of 0.5 to 40%, 1 to 30%, 5-40%, 10-35%, 10-30%, 15-30.%, 20-30% or 25-30% by weight or by volume. In one embodiment the pharmaceutical or otherwise active agent is kava which is present in an amount of 0.5 to 40%, 1 to 30%, 5-40%, 10-35%, 10-30%, 15-30.%, 20-30% or 25-30% by weight or by volume.

The topical composition can include a wax which is present in an amount of 5 to 95%, 10 to 80%, 15-80%, 20-75%, 25-70%, 25-50%, 30-50% or 35-50% by weight or by volume.

The topical composition can include an oil which is present in an amount of 1 to 40%, 1 to 30%, 5-40%, 10-35%, 10-30%, 15-30.%, 20-30% or 25-30% by weight or by volume.

The composition can comprise from about 0.1% to about 10% by weight or by volume of the additive, for example, 0.1-1.0%, 0.1-0.9%, 0.1-0.8%, 0.1-0.7%, 0.1-0.6%, 0.1-0.5%, 0.1-0.3%, 0.1-0.2%, 1-5%, 2-5%, 2-6%, 3-8%, 4.0-9%, 5-8%, 6-9%, 5-10%, 7-10%, or 9-10%, by weight or by volume.

In one embodiment, the composition comprises a cannabinoid present in an amount of about 1% to about 30% weight percent of the composition, lidocaine or benzocaine present in an amount of 1% to 20% of the composition, Manuka honey present in an amount of about 1% to about 20% weight percent of the composition, thickener in an amount of about 20% to about 96% and oil in an amount of about 1% to about 40% weight percent of the composition. In one embodiment, the composition comprises Stachys byzantine of about 1% to about 40% weight percent of the composition, lidocaine or benzocaine present in an amount of 1% to 20% of the composition, Manuka honey present in an amount of about 1% to about 20% weight percent of the composition, thickener in an amount of about 20% to about 96% and oil in an amount of about 1% to about 40% weight percent of the composition, optionally the compositions can include 1% to 30% weight percent of the composition of a cannabinoid and optionally the composition can include 1% to 30% weight percent of the composition of kava.

In one embodiment the topical composition comprises coconut oil, natural cocoa butter, bees Wax, olive oil, avocado Oil, mineral oil, sunflower seed oil, green Tea, CBG Oil, CBD Oil, 4% Lidocaine and optionally aloe vera gel.

In one embodiment the topical composition comprises coconut oil, cocoa butter, bees wax, olive oil, avocado oil, green tea leaves, lidocaine 4%, CBD oil, and CBG oil.

In one embodiment the topical composition comprises shea butter, coconut oil, vitamin E oil, Stachys byzantine, benzocaine, Manuka honey, and optionally kava, capsaicin, lavender, aloe vera gel, eucalyptus oil, and peppermint.

The cannabinoid and pharmaceutical according to the present invention can include pharmaceutically acceptable forms thereof, including isomers such as diastereomers and enantiomers, salts, solvates, and polymorphs, as well as racemic mixtures.

The composition can be formulated by mixing two or more of the ingredients at room temperature. In one embodiment, all ingredients of the composition can be mixed at room temperature. Alternatively, the ingredients during mixing are heated from about 50□C to 100□C, from about 50□C to 95 □C, 50□C to 85□C 50 or from about 50□C to 100□C.

Methods of Use

The topical composition according to the present invention can be administered to or topically applied to a human or other animal subject. The present invention further provides a use of comprising a therapeutically-effective amount of at least one cannabinoid and at least one pharmaceutical active component, wherein the at least one pharmaceutical active component improves health beyond benefits provided by the at least one cannabinoid alone, in a topical composition, for reducing pain and inflammation in a subject. The topical composition is preferably as defined herein.

The invention is further illustrated in the following non-limiting examples. The examples are given solely for the purposes of illustration and are not to be construed as limitations of the present disclosure.

### Example 1

An example composition with ingredient ranges for a topical composition to be used as a rub can be formulated as shown in Table 1.

TABLE 1
Ingedient

| | | |
|---|---|---|
| Coconut Oil | 453,590 | mg |
| Cocoa Butter | 226,795 | mg |
| Bees Wax | 453,590-566,990 | mg |
| Olive Oil | 226,795 | mg |
| Avocado Oil | 56,700-226,795 | mg |
| Green Tea Leaves | 56,700 | mg |
| Lidocaine 4% | 500-115,000 | mg |
| CBD Oil | 375 | mg |
| CBG Oil | 500 | mg |

### Example 2

An example composition with ingredient ranges for a topical composition to be used as a rub can be formulated as shown in Table 2.

TABLE 2
Ingredient

| | | |
|---|---|---|
| Shea Butter | 226,795 | mg |
| Coconut Oil | 453,590 | mg |
| Vitamin E Oil | 226,795 | mg |
| Stachys Byzantina | 375-115,000 | mg |
| Benzocaine | 500-115,000 | mg |
| Manuka Honey | 56,700-227,000 | mg |
| Capsaicin | 375-115,000 | mg |
| Lavender | 375-115,000 | mg |
| Aloe Vera Gel | 56,700-453,590 | mg |
| Eucalyptus Oil | 375-115,000 | mg |
| Peppermint | 375-115,000 | mg |

It is to be understood that the above described embodiments are illustrative of only a few of the many possible specific embodiments, which can represent applications of the principles of the invention. Numerous and varied other arrangements can be readily devised in accordance with these principles by those skilled in the art without departing from the scope of the invention.

What is claimed is:

1. A topical composition comprising a therapeutically-effective amount of at least one cannabinoid, Manuka honey and at least one pharmaceutical active component comprising a pain reducing active, wherein the at least one pharmaceutical active component and the Manuka honey improve pain reduction and inflammation benefits beyond benefits provided by the at least one cannabinoid alone.

2. The topical composition of claim 1 wherein the at least one pharmaceutical active component is a local anesthetic.

3. The topical composition of claim 1 wherein the at least one pharmaceutical active component is selected from the group consisting of lidocaine, benzocaine, bupivacaine, mepivacaine and articaine.

4. The topical composition of claim 1 wherein the at least one pharmaceutical active component is lidocaine.

5. The topical composition of claim 1 wherein the pharmaceutical active component further comprises a steroid to treat a skin condition.

6. The topical composition of claim 1 wherein the pharmaceutical active component further comprises one or more of a steroid selected from hydrocortisone, clobetasol and halobetasol.

7. The topical composition of claim 1 further comprising a vitamin or herbal supplement.

8. The topical composition of claim 7 wherein the vitamin is selected from the group consisting of E, D, and B12.

9. The topical composition of claim 7 wherein the herbal supplement is selected from the group of cayenne, capsicum, capsaicin, and capsaicinoid compounds.

10. The topical composition of claim 1 further comprising one or more extracts selected from the group consisting of peppermint oil, tea tree oil, eucalyptus oil, chamomile oil, neem oil, lavender oil, garlic extract, neem extract, marigold extract and oregano extract.

11. The topical composition of claim 1 wherein the at least one cannabinoid is selected from cannabidiol (CBD), cannabigerol (CBG), Cannabidiphorol (CBDP) and cannabichromene (CBC).

12. The topical composition of claim 1 wherein the cannabinoid is in liquid or solid form.

13. The topical composition of claim 1 wherein the cannabinoid is cannabidiol (CBD) oil and cannabigerol (CBG) oil.

14. The topical composition of claim 1 further comprising one or more adaptogenic herbs selected from menthol, lavender, rose hip, rose petals, fennel, and tea leaves.

15. The topical composition of claim 1 wherein the composition further comprises one or more additives selected from diluents, preservatives, surfactants, thickening agents, emulsifiers, humectants, pigments, emollients, and mixtures thereof.

16. The topical composition of claim 1 further comprising one or more additives selected from the group comprising natural cocoa butter, bees wax, shea butter, candelilla vegan wax, wax, aloe vera gel, petroleum jelly, coconut oil, olive oil, avocado oil, mineral oil, and sunflower seed oil.

17. The topical composition of claim 1 further comprising at least one antifungal agent selected from the group consisting of ketoconazole, bifonazole, butoconazole, clotrimazole, econazole, fenticonazole, isoconazole, luliconazole, miconazole, omoconazole, oxiconazole, sertaconazole, sulconazole, tioconazole, albaconazole, fluconazole, isavuconazole, itraconazole, posaconazole, ravuconazole, terconazole, and voriconazole,

18. The topical composition of claim 1 further comprising at least one sunscreen agent selected from the group consisting of aminobenzoic acid (PABA), avobenzone, cinoxate, dioxybenzone, ensulizole, homosalate, meradimate, octinoxate, octisalate, octocrylene, padimate O, sulisobenzone, oxybenzone, trolamine salicylate, ethylhexyl methoxycinnamate, octyl methoxycimnamate, octyl salicylate, phenylbenzimidazole sulfonic acid, titanium dioxide, trolamine dioxide and zinc oxide.

19. The topical composition of claim 1 wherein the at least one pharmaceutical active component further comprises kava or Stachys byzantina.

20. A topical composition comprising a therapeutically-effective amount of at least one cannabinoid, Manuka honey, lidocaine, thickener and oil, wherein the cannabinoid is present in an amount of about 1% to about 30% weight percent of the composition, the Manuka honey is present in an amount of about 1% to about 20% weight percent of the composition, the lidocaine is present in an amount of about 1% to about 20% weight percent of the composition, the thickener in an amount of about 20% to about 96% weight percent of the composition and the oil is present in an amount of about 1% to about 40% weight percent of the composition.

21. The topical composition of claim 20 wherein the cannabinoid is cannabidiol (CBD) oil and cannabigerol (CBG) oil.

22. The topical composition of claim 1 further comprising butter.

23. The topical composition of claim 20 further comprising butter.
